(19) United States
(12) Patent Application Publication
(10) Pub. No.: US 2005/0250733 A1
(43) Pub. Date: Nov. 10, 2005

(54) Purified, moderately esterified polyol polyester fatty acid compositions

(75) Inventor: Jared John Schaefer, Wyoming, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/841,179

(22) Filed: May 7, 2004

(51) Int. Cl. A61K 31/7024

(52) U.S. Cl. 514/53; 536/110

(57) ABSTRACT

Processes for the production of purified, moderately esterified polyol fatty acid polyesters and the compositions derived from those processes. The purified, moderately esterified polyol fatty acid polyesters are particularly well suited for use in a variety of food, beverage, pharmaceutical, and cosmetic applications. Purified, moderately esterified polyol fatty acid polyester composition containing less than about 5% polyol; substantially free of residual solvent; less than about 700 ppm of lower alkyl esters; less than about 2% of a soap and free fatty acid mixture; less than about 1% of ash; and an acid value of less than about 2.

### CROSS-REFERENCE TO RELATED APPLICATIONS

Under 35 USC § 120, this application claims benefit of U.S. patent application Ser. No. 10/156,479, filed May 28, 2002; U.S. patent application Ser. No. 10/156,437, filed May 28, 2002; and U.S. patent application Ser. No. 10/156,476 filed May 28, 2002.

### BRIEF DESCRIPTION OF THE INVENTION

This invention relates to the production of moderately esterified polyol fatty acid polyesters. More particularly, this invention relates to purified, moderately esterified polyol fatty acid polyesters derived from processes that include aqueous and alcohol based purification steps.

### BACKGROUND OF THE INVENTION

As a result of their physical properties, moderately esterified polyol fatty acid polyesters are useful in a variety of applications. In particular, moderately esterified polyol fatty acid polyesters are well suited for use in various laundry, textile, food, and cosmetic compositions.

Various techniques are available for the synthesis of these moderately esterified polyol fatty acid polyesters. Although such techniques have known utilities, they often suffer from several deficiencies, most notable of which include poor reaction control and/or the need for expensive and complex purification techniques.

Additionally, such processes are often unable to accurately predict and consistently control the exact composition of the finished product without the use of complex sampling and/or control modification procedures throughout the reaction. Such processes also often suffer from an inability to accurately control the average degree of esterification in the final moderately esterified polyol polyester compositions. Moreover, the moderately esterified polyol polyester compositions produced from such synthesis techniques typically contain unacceptable levels of undesirable impurities, such as solvent, polyol, lower alkyl esters, ash, soap, free fatty acids, and/or other unwanted reaction byproducts. Finally, such processes often require the use of either a reaction solvent or an emulsifying agent (such as soap) to promote contact between the otherwise insoluble reactants. During the process, such solvents or emulsifying agents must then be removed or it will exist as an impurity in the final product. The additional removal steps create additional costs and time demands.

The limitations and difficulties described above have heretofore constrained the industrial applicability and cost effective commercialization of these moderately esterified compounds in various laundry, textile, food, lubricant, and cosmetic applications.

Accordingly, there is a need to provide processes for the synthesis of purified, moderately esterified polyol polyesters that allow for the production of polyol polyesters with the improved purity necessary for widespread incorporation into a variety of industrial and commercial applications. Additionally, there is a need to provide purified, moderately esterified polyol polyester compositions with the improved purity that is sufficient to be used in a variety of industrial and commercial applications. There is also a need to provide processes for the production of purified moderately esterified polyol polyesters that are efficient, cost effective, and typically require less purification than those now known and employed in the art. In addition, there is a need to provide processes that result in products with a degree of esterification that is controllable and/or reproducible. There is also a need to provide processes for the production of purified moderately esterified polyol polyesters using only polyols, highly esterified polyol polyesters, moderately esterified polyol polyesters, and a catalyst without the use of a reaction solvent or emulsifying agent which exist as impurities in the final product.

### SUMMARY OF THE INVENTION

The present invention relates to processes for the production of purified, moderately esterified polyol fatty acid polyesters and the compositions made from those processes. More particularly, this invention relates to processes for preparing moderately esterified polyol fatty acid polyesters that include aqueous and alcohol based purification processes.

The purified, moderately esterified polyol fatty acid polyesters of the present invention are particularly well suited for use in a variety of laundry, textile, food, lubricant, and cosmetic applications, and comprise less than about 5% polyol; less than about 700 ppm of lower alkyl esters; less than about 2% of a soap and free fatty acid mixture; less than about 1% of ash; an acid value of less than about 2; and are substantially free of residual reaction solvent.

In one embodiment, the purified moderately esterified polyol polyester is a purified moderately esterified sucrose polyester containing less than about 2% sucrose; no residual reaction solvent; less than about 600 ppm of lower alkyl esters; less than about 1% of a soap and free fatty acid mixture; less than about 0.5% of ash; and an acid value of less than about 1.

### DETAILED DESCRIPTION OF THE INVENTION

The present invention encompasses esterification processes for the production of moderately esterified polyol fatty acid polyesters, in particular highly purified, moderately esterified polyol fatty acid polyesters. The present invention will now be described in detail with reference to specific embodiments.

A. Definitions

Various publications and patents are referenced throughout this disclosure. All references cited herein are hereby incorporated by reference. Unless otherwise indicated, all percentages and ratios are calculated by weight. All percentages and ratios are calculated based on the total dry composition unless otherwise indicated.

All component or composition levels are in reference to the active level of that component or composition, and are substantially free of impurities, for example, residual solvents or by-products, which may be present in commercially available sources.

Referred to herein are trade names for components including various ingredients utilized in the present invention. The inventors herein do not intend to be limited by materials under a certain trade name. Equivalent materials (e.g., those obtained from a different source under a different name or catalog number) to those referenced by trade name may be substituted and utilized in the compositions, kits, and methods herein.

As used herein, and unless otherwise indicated, the use of a numeric range to indicate the value of a given variable is not intended to be limited to just discrete points within that stated range. One of ordinary skill in the art will appreciate that the use of a numeric range to indicate the value of a variable is meant to include not just the values bounding the stated range, but also all values and sub-ranges contained therein. By way of example, consider variable X that is disclosed as having a value in the range of A to B. One of ordinary skill in the art will understand that variable X is meant to include all integer and non-integer values bounded by the stated range of A to B. Moreover, one of ordinary skill in the art will appreciate that the value of the variable also includes all combinations and/or permutations of sub-ranges bounded by the integer and non-integer values within and including A and B.

As used herein, the term “moderately esterified polyol polyester” is intended to include those esters of the polyol having a degree of esterification in excess of the degree of esterification of the polyol, but less than the degree of esterification of the highly esterified polyol fatty acid polyester. As used herein, the term “degree of esterification” refers to the average percentage of hydroxyl groups of a polyol composition that have been esterified.

In one embodiment of the present invention the polyol is sucrose having eight hydroxyl groups. The moderately esterified sucrose polyester preferably has a degree of esterification of from about 40% to about 80%. As used herein the degree of esterification calculation does not include non-esterified polyol compounds that may be present. As will be appreciated by the ordinarily skilled artisan, the degree of esterification of an esterified polyol polyester may also be expressed by the polyol polyester's I-bar (\overline{I}) value. As used herein, the term “I-bar (\overline{I})” is defined as the molar average number of hydroxyl groups of the polyol that have been esterified.

In one embodiment of the present invention the polyol is sucrose having eight hydroxyl groups. The moderately esterified sucrose polyester preferably has an I-bar value in the range of from about 3.2 to about 6.4. As used herein the I-bar calculation does not include non-esterified polyol compounds that may be present.

In the description of the invention various embodiments and/or individual features are disclosed. As will be apparent to the ordinarily skilled practitioner, all combinations of such embodiments and features are possible and can result in preferred executions of the present invention.

B. Processes for Synthesizing Purified, Moderately Esterified Polyol Polyester Fatty Acid Compositions

In general, the processes for the preparation of purified, moderately esterified polyol fatty acid polyesters of the present invention comprise the steps of forming an initial reaction product from an initial reaction mixture; optionally neutralizing any remaining reaction catalyst; optionally forming a secondary reaction product to recover residual reaction components (e.g. solvent, if used) via such processes as evaporation, purifying the reaction product to remove any impurities and/or unreacted components; and optionally drying the purified reaction product. Preferably, no reaction solvent is used during the preparation process so that there is no reaction solvent residual to be removed.

i) Initial Reaction Product

An initial reaction product is formed by reacting an initial reaction mixture in an inert atmosphere, for a period of time in the range of from about 30 minutes to about 6 hours, and at a temperature in the range of from about 80° C. to about 140° C.

The initial reaction mixture comprises a polyol portion, a highly esterified polyol fatty acid polyester, a moderately esterified polyol polyester, and a catalyst. Preferably, the molar ratio of the catalyst to the highly esterified polyol fatty acid polyester is in the range of from about 0.01:1 to about 10:1, more preferably in the range of from about 0.1:1 to about 5:1, yet more preferably from about 0.25:1 to about 1:1, most preferably in the range of from about 0.4:1 to about 0.6:1. Preferably, the molar ratio of polyol, highly esterified polyol fatty acid polyester, and moderately esterified polyol fatty acid polyester should be chosen such that the final ratio of total fatty acid esters to total polyol backbones added is in the range of from about 3.2:1 to about 6.4:1. Those skilled in the art will understand the flexibility available in the ratios of polyol to highly esterified polyol polyester, polyol to moderately esterified polyol polyester, and highly esterified polyol polyester to moderately esterified polyol polyester that are able to provide a mixture with a total ratio of fatty acid esters to polyol backbones of from about 3.2:1 to 6.4:1. One skilled in the art will also appreciate the term “total fatty acid esters” to mean the total fatty acid ester chains contributed from the highly esterified polyol polyesters and moderately esterified polyol polyesters and the term “total polyol backbones” to mean the total polyol molecules contributed from the polyol, the highly esterified polyol polyester, and the moderately esterified polyol polyester. In the case of the “total fatty acid esters”, the number of fatty acid esters contributed by the highly esterified polyol polyester and the moderately esterified polyol polyester will depend on the average degree of esterification of the molecules, whereas the “total polyol backbones” contributed by the polyol, the highly esterified polyol polyester, and the moderately esterified polyol polyester will depend only on the amounts of each species present, as each contributes one polyol backbone per molecule. The examples provided will provide further clarity on this discussion.

In one embodiment of the present invention the polyol is sucrose, the highly esterified polyol fatty acid polyester is sucrose polyester with a degree of esterification of about 95%, and the moderately esterified polyol fatty acid polyester is sucrose ester with a degree of esterification of about 50%.

As used herein, the term “polyol” is intended to include any aliphatic or aromatic compound containing at least two free hydroxyl groups. In practicing the processes disclosed herein, the selection of a suitable polyol is simply a matter of choice. For example, suitable polyols may be selected from the following classes: saturated and unsaturated straight and branched chain linear aliphatic; saturated and unsaturated cyclic aliphatic, including heterocyclic aliphatic; or mononuclear or polynuclear aromatics, including heterocyclic aromatics. Carbohydrates and glycols are exemplary polyols. Especially preferred glycols include glycerin. Monosaccharides suitable for use herein include, for example, mannose, galactose, arabinose, xylose, ribose, apiose, rhamnose, psicose, fructose, sorbose, tagitose, ribulose, xylulose, and erythrulose. Oligosaccharides suitable for use herein include, for example, maltose, kojibiose, nigerose, cellobiose, lactose, melibiose, gentiobiose, turanose, rutinose, trehalose, sucrose and raffinose. Polysaccharides suitable for use herein include, for example, amylose, glycogen, cellulose, chitin, inulin, agarose, zylans, mannan and galactans. Although sugar alcohols are not carbohydrates in a strict sense, the naturally occurring sugar alcohols are so closely related to the carbohydrates that they are also preferred for use herein. The sugar alcohols most widely distributed in nature and suitable for use herein are sorbitol, mannitol and galactitol.

Particular classes of materials suitable for use herein include monosaccharides, disaccharides and sugar alcohols. Other classes of materials include sugar ethers and alkoxylated polyols, such as polyethoxy glycerol.

In one embodiment of the present invention the polyol has on average at least four, preferably at least about 5, more preferably about 8 hydroxyl groups capable of being esterified per polyol molecule.

Suitable esterified epoxide-extended polyols include esterified propoxylated glycerols prepared by reacting a propoxylated glycerol having from 2 to 100 oxypropylene units per glycerol with C₁₀-C₂₄ fatty acids or with C₁₀-C₂₄ fatty acid esters, as described in U.S. Pat. Nos. 4,983,329 and 5,175,323, respectively, and esterified propoxylated glycerols prepared by reacting an epoxide and a triglyceride with an aliphatic polyalcohol, as described in U.S. Pat. No. 5,304,665 or with an alkali metal or alkaline earth salt of an aliphatic alcohol, as described in U.S. Pat. No. 5,399,728. Other polyols include acylated propylene oxide-extended glycerols having a propoxylation index of above about 2, preferably in the range of from about 2 to about 8, more preferably about 5 or above, wherein the acyl groups are C₈-C₂₄, preferably C₁₄-C₁₈, compounds, as described in U.S. Pat. Nos. 5,603,978 and 5,641,534 and fatty acid-esterified propoxylated glycerols, as described in U.S. Pat. Nos. 5,589,217 and 5,597,605.

Other suitable esterified epoxide-extended polyols include esterified alkoxylated polysaccharides. Preferred esterified alkoxylated polysaccharides are esterified alkoxylated polysaccharides containing anhydromonosaccharide units, more preferred are esterified propoxylated polysaccharides containing anhydromonosaccharide units, as described in U.S. Pat. No. 5,273,772.

The polyol has a degree of esterification less than the degree of esterification of both the moderately esterified polyol polyester and the highly esterified polyol fatty acid polyester. The polyol portion may be a single type or class of polyol (e.g., sucrose) or may alternatively be a blend of two or more types or classes of polyols (e.g., a sugar alcohols, such as sorbitol; monosaccharides, such as fructose; and oligosaccharides, such as maltose).

As used herein, the term “highly esterified polyol fatty acid polyester” is intended to include those esters of a polyol with a degree of esterification in excess of the degree of esterification of both the polyol and the moderately esterified polyol polyester. In one embodiment of the invention the highly esterified polyol polyester has a degree of esterification of at least about 70%, while in yet another embodiment the highly esterified polyol polyester has a degree of esterification of at least about 90%, preferably at least about 95%.

A variety of processes are known in the art for the synthesis of highly esterified polyol fatty acid polyesters that are suitable for use in the processes of the present invention. Examples of such processes are detailed in U.S. Pat. No. 3,963,699, to Rizzi et al., disclosing a solvent-free transesterification process in which a mixture of a polyol (such as sucrose), a fatty acid lower alkyl ester (such as a fatty acid methyl ester), an alkali metal fatty acid soap, and a basic catalyst is heated to form a homogenous melt. Excess fatty acid lower alkyl ester is added to the melt to form the higher polyol fatty acid polyesters. The polyesters are then separated from the reaction mixture by any of the routinely used separation procedures. Additional suitable processes include U.S. Pat. No. 4,517,360, to Volpenhein et al.; U.S. Pat. No. 5,422,131, to Elsen et al.; U.S. Pat. No. 5,648,483, to Granberg et al.; U.S. Pat. No. 5,767,257, to Schafermeyer et al., and U.S. Pat. No. 6,261,628, to Howie et al.

In one embodiment of the present invention, the highly esterified polyol fatty acid polyesters are sucrose fatty acid polyesters, having an average of at least 4 fatty acid groups per molecule. In another embodiment of the invention, the highly polyol fatty acid polyester is sucrose fatty acid polyester having an average of at least 5 fatty acid groups per molecule, while in another embodiment the sucrose fatty acid polyesters have an average of from about 5 to about 8 fatty acid groups per molecule. In yet another embodiment, the polyol polyester is a sucrose polyester wherein at least about 75% of the sucrose polyester comprises octaester.

In one embodiment of the present invention, the moderately esterified polyol fatty acid polyesters are sucrose fatty acid esters, having an average of from about 3.2 to 6.4 fatty acid groups per molecule. In another embodiment of the invention, the moderately esterified polyol fatty acid polyesters are sucrose fatty acid ester having an average of from about 4 to 5 fatty acid groups per molecule.

The fatty acid chains of the highly esterified polyol fatty acid polyesters and the moderately esterified polyol polyester may be branched, linear, saturated, unsaturated, hydrogenated, unhydrogenated, or mixtures thereof. The fatty acid chains of the fatty acid esters have from about 6 to about 30 total carbon atoms. As used herein, reference to a fatty acid compound having fatty acid chains of a particular length is intended to mean that a majority of the fatty acid chains, i.e., greater than 50 mol % of the fatty acid chains, have the stated length. In a more specific embodiment, the fatty acid compounds have greater than about 60 mol %, and more specifically greater than about 75 mol %, of fatty acid chains of the stated length. As used herein “fatty acid ester” is intended to include fatty acid esters in which the fatty acid chains have a total of from about 2 to about 28, typically from about 8 to about 22, carbon atoms. The fatty acid esters may be branched, unbranched, saturated, unsaturated, hydrogenated, unhydrogenated, or mixtures thereof.

In one embodiment of the present invention, the fatty acid chains of the polyester may be branched or linear and may be formed from fatty acid esters having fatty acid chains of from about 8 to about 26 total carbon atoms. In yet another embodiment, the fatty acid chains of the fatty acid ester have from about 16 to about 22 total carbon atoms.

In one embodiment of the present invention, the fatty acid chains of the highly esterified polyol polyester are substantially the same type of fatty chains as the moderately esterified polyol polyester. In another embodiment, the fatty acid chains of the highly esterified polyol polyester and the moderately esterified polyol polyester are different, i.e. the chains on the highly esterified polyol polyester can be branched or a different carbon length than the moderately esterified polyol polyester.

Other suitable polyol fatty acid polyesters are esterified linked alkoxylated glycerins, including those comprising polyether glycol linking segments, as described in U.S. Pat. No. 5,374,446 and those comprising polycarboxylate linking segments, as described in U.S. Pat. Nos. 5,427,815 and 5,516,544.

Additional suitable polyol fatty acid polyesters are esterified epoxide-extended polyols of the general formula P(OH)_{A+C}(EPO)_{N}(FE)_{B} wherein P(OH) is a polyol, A is from 2 to about 8 primary hydroxyls, C is from about 0 to about 8 total secondary and tertiary hydroxyls, A+C is from about 3 to about 8, EPO is a C₃-C₆ epoxide, N is a minimum epoxylation index average number, FE is a fatty acid acyl moiety and B is an average number in the range of greater than 2 and no greater than A+C, as described in U.S. Pat. No. 4,861,613. The minimum epoxylation index average number has a value generally equal to or greater than A and is a number sufficient so that greater than 95% of the primary hydroxyls of the polyol are converted to secondary or tertiary hydroxyls. Preferably the fatty acid acyl moiety has a C₇-C₂₃ alkyl chain.

The highly esterified polyol fatty acid polyester may be comprised of a single type or class of polyol polyester (e.g., sucrose) or may alternatively be a blend of two or more types or classes of polyol polyesters (e.g. sugar alcohols, such as sorbitol; monosaccharides, such as fructose; and oligosaccharides, such as maltose). The polyol backbones of the highly esterified polyol fatty acid polyesters (e.g., sucrose in a highly esterified sucrose fatty acid polyester) may be the same backbone as the polyol, or may optionally be different.

The moderately esterified polyol fatty acid polyester may be comprised of a single type or class of polyol polyester (e.g., sucrose) or may alternatively be a blend of two or more types or classes of polyol polyesters (e.g. sugar alcohols, such as sorbitol; monosaccharides, such as fructose; and oligosaccharides, such as maltose). The polyol backbones of the moderately esterified polyol fatty acid polyesters (e.g., sucrose in a moderately esterified sucrose fatty acid polyester) may be the same backbone as the polyol, or may optionally be different.

In one embodiment of the present invention the polyol is sucrose, the highly esterified polyol fatty acid polyester is predominantly (i.e., in excess of about 95%, preferably in excess of about 98%, more preferably in excess of about 99%) comprised of sucrose fatty acid polyester, and the moderately esterified polyol polyester is predominantly comprised of sucrose fatty acid esters with from 3.2 to 6.4 fatty chains esterified to sucrose. In another embodiment the polyol is glucose, the highly esterified polyol fatty acid polyester is sucrose fatty acid polyester, and the moderately esterified polyol polyester is predominantly comprised of sucrose fatty acid esters with from 3.2 to 6.4 fatty chains esterified to sucrose. In yet another embodiment, the polyol is sucrose, the highly esterified fatty acid polyester is comprised of sucrose fatty acid polyester and a highly esterified epoxide-extended polyol polyester, and the moderately esterified polyol polyester is comprised of sorbitan with from 1-3 fatty chains esterified to sorbitan

Suitable basic compounds to be used as basic reaction catalysts include alkali metals such as sodium, lithium and potassium; alloys of two or more alkali metals such as sodium-lithium and sodium-potassium alloys; alkali metal hydrides, such as sodium, lithium and potassium hydride; alkali metal lower (C₁-C₄) alkyls such as butyl-lithium; and alkaline metal alkoxides of lower (C₁-C₄) alcohols, such as lithium methoxide, potassium t-butoxide, potassium methoxide, and/or sodium methoxide. Other suitable basic compounds include carbonates and bicarbonates of alkali metals or alkaline earth metals. Preferred classes of basic catalysts include potassium carbonate, sodium carbonate, barium carbonate, or mixtures of these compounds having particle sizes that are less than about 100 microns, preferably less than about 50 microns. These preferred catalysts could be used in admixture with the more conventional basic catalysts, described above. Potassium carbonate and/or potassium methoxide are also preferred catalysts. These catalysts are further disclosed in U.S. Pat. No. 4,517,360, to Volpenhein et al.

Applicants have found that during the initial reaction phase it is preferable that the initial reaction mixture be as homogeneous as possible. Although it is preferred that the initial reaction mixture not include a solvent, small amounts of solvent may be used. When used, the initial reaction mixture comprises less than about 10% solvent, by weight of the initial reaction mixture, alternatively, less than 5%, alternatively, less than 4% of solvent. Where utilized, suitable solvents include dimethyl sulfoxide, n-methyl formamide, dimethyl sulfate, formamide, and mixtures thereof. A homogenous initial reaction mixture can be achieved by selection of appropriate reaction mixture ingredients that dissolve in the presence of the middle polyol fatty acid ester, and the selected solvent, if a solvent is used.

If the preferred degree of homogeneity is not readily achieved upon the admixing of the initial reaction mixture components, either by virtue of the ingredients or various other processing parameters selected, a sufficient amount of agitation may be applied during the initial reaction phase to form an approximately homogeneous mixture or emulsion. Agitation should be applied for a period of time necessary to maintain homogeneity throughout the duration of the initial reaction. Once agitation has been applied for a period of time necessary to assure homogeneity of the reactants throughout the reaction, further application of agitation may be continued, discontinued, or varied in force.

As used herein the term, “a sufficient amount of agitation” is defined as the level of agitation necessary to ensure that reaction components (e.g., the initial reaction mixture) do not separate into discrete phases for a period of time in excess of about 10 seconds, preferably in excess of about 20 seconds, more preferably in excess of about 30 seconds, more preferably in excess of about 45 seconds, most preferably in excess of about 60 seconds, following discontinuation of the agitation. Preferably, agitation is applied during the reaction for a period of time sufficient to ensure that the degree of esterification of the highly esterified polyol polyester fatty acid is reduced to below about 95%, preferably below about 90%, more preferably below about 80%.

In one embodiment of the present invention a heterogeneous initial reaction mixture comprises sucrose, a highly esterified sucrose fatty acid ester with a degree of esterification of about 95%, a moderately esterified sucrose fatty acid ester with a degree of esterification of about 50%, and a potassium carbonate catalyst. Agitation is applied by use of a rotating impeller. The degree of agitation necessary to ensure a suitable degree of homogeneity throughout the reaction is quantified by a Weber Number in the range of from about 2000 to about 20,000, operating for a period of time in the range of from about 10 minutes to about 6 hours. In another embodiment the degree of agitation necessary to ensure suitable homogeneity is quantified by a Weber Number of about 10,000, applied for approximately 60 minutes. In yet another embodiment the agitation is quantified by a Weber Number of about 9,000 applied for the entire duration of a 120-minute reaction time.

As used herein, any device capable of inducing motion in the fluid reaction mixtures over a range of viscosities, thus effecting a dispersion of the components, is a suitable agitator for use in the processes of the present invention. Examples of suitable agitators include impellers, paddles, kneaders, helical rotors, single sigma blade, double sigma blades, screw-type agitators, ribbon agitators, and mixtures thereof.

As used herein, the “Weber Number” is a dimensionless number intended to provide a system independent measure of the agitation force applied to a reaction mixture. The Weber Number is defined by Equation 1.

$$\frac{\begin{matrix}( Density of the Continuous Phase ) \times \\ {( RPM of the Impellor )}^{2} \times \\ {( Diameter of the Impellor )}^{3}\end{matrix}}{\begin{matrix}Interfacial Tension between the Continuous \\ and Discontinuous Phases\end{matrix}} . \qquad Equation 1$$

ii) Catalyst Neutralization

Optionally, any catalyst remaining subsequent to the formation of the initial reaction product may be neutralized with an acid. Applicants have hereby found that neutralization of the remaining catalyst reduces the risk of saponification and base catalyzed hydrolysis reactions during aqueous purification, both of which adversely impact the purity of the moderately esterified polyol fatty acid compositions.

To effectively neutralize any residual catalyst, a sufficient amount of an acid is added to the initial reaction product such that the molar ratio of the acid to total catalyst is in the range of from about 0.01:1 to about 1:1, preferably in the range of from about 0.1:1 to about 0.8:1, more preferably in the range of from about 0.6:1 to about 0.8:1. Examples of acids suitable for use in neutralizing any residual base catalyst include those acids selected from hydrochloric, phosphoric, chromic, iodic, benzoic, hydrofluoric, sulfuric, sulfurous, acetic, formic, nitric, and mixtures thereof.

iii) Secondary Reaction Product

In one embodiment, a secondary reaction product may be formed subsequent to the formation of the initial reaction product. The primary purpose for forming the secondary reaction product is to recover various initial reaction mixture components, such as any reaction solvent, that are no longer required for the remaining purification processes. Preferably, no reaction solvent is used other than the middle polyol fatty acid polyester and therefore the secondary reaction product may be unnecessary. However, if small amounts of reaction solvent are used, removal of the solvent by formation of the secondary reaction product reduces the amount of solvent present in the final moderately esterified polyol fatty acid polyester compositions.

The secondary reaction product is formed by reacting the initial reaction product at a pressure in the range of from about 0.01 mmHg to about 760 mmHg, preferably in the range of from about 0.1 mmHg to about 20 mmHg, more preferably in the range of from about 0.1 mmHg to about 10 mmHg, most preferably in the rang of from about 0.1 mmHg to abut 5 mmHg, and for a period of time in the range of from about 30 minutes to about 4 hours.

In one embodiment of the present invention the desired reaction pressure dictates the temperature at which the secondary reaction product is formed. In another embodiment of the invention the desired reaction temperature dictates the reaction pressure to be employed. Preferably, when formed, the secondary reaction product is formed at the temperature-pressure combination at which distillation of any solvent used in the initial reaction mixture occurs.

In one embodiment of the present invention, the step of neutralizing any remaining catalyst is performed subsequent to the formation of the initial reaction product, but prior to the formation of a secondary reaction product. In another embodiment the secondary reaction product is formed subsequent to the formation of the initial reaction product, though prior to the neutralization of remaining catalyst. In yet another embodiment, the remaining catalyst is neutralized with an acid without the formation of a secondary reaction product. In yet another embodiment the secondary reaction product is formed, while the remaining catalyst is not neutralized.

iv) Purification

(a) Solvent Free Aqueous Purification Processes

The reaction products of the present invention may be purified by an aqueous purification process, via application of a water washing solution. Applicants have found that in order to obtain moderately esterified polyol polyester compositions with the requisite degree of purity, the aqueous purification process should be free of any solvents that would adversely affect the finished product purity requirement for the composition's intended use. As any solvent added after formation of the initial reaction product must ultimately be removed via a purification process, it is particularly preferred that the aqueous purification process be a solvent free purification process.

The water washing solution comprises about 100% water, which may optionally be distilled, purified, or de-ionized. The water washing solution may optionally comprise from about 0.1% to about 5% of a salt and from about 95% to about 99.9% water. The water washing solution is applied over a period of time in the range of from about 2 minutes to about 30 minutes, preferably in the range of from about 5-10 minutes. The weight ratio of the water washing solution to the initial weight of the reaction product to be purified (e.g. initial reaction product; secondary reaction product; acid neutralized initial reaction product; or acid neutralized secondary reaction product) is in the range of from about 0.01:1 to about 1:1, preferably in the range of from about 0.05:1 to about 0.5:1, more preferably in the range of from about 0.1:1 to about 0.3:1. The temperature of the water washing solution is in the range of from about 20° C. to about 100° C., and the temperature of the reaction product to be purified is in the range of from about 20° C. to about 100° C. Preferably the temperature of the water washing solution is in the range of from about 20° C. to about 60° C. when the majority of the fatty acid esters are unsaturated, and in the range of from about 40° C. to about 80° C. when the majority of the fatty acid esters are saturated.

Examples of salts suitable for use in the present invention include salts selected from calcium salts, magnesium salts, barium salts, sodium salts, potassium salts, cesium salts, and mixtures thereof. Preferred salts for use in the present invention include salts selected from lithium chloride, lithium bromide, lithium iodide, lithium sulfate, calcium chloride, calcium bromide, calcium iodide, calcium sulfate, magnesium chloride, magnesium bromide, magnesium iodide, magnesium sulfate, barium chloride, barium bromide, barium iodide, barium sulfate, sodium chloride, sodium bromide, sodium iodide, sodium sulfate, potassium chloride, potassium bromide, potassium iodide, potassium sulfate, cesium chloride, cesium bromide, cesium iodide, cesium sulfate, and mixtures thereof. Salts selected from calcium chloride, calcium bromide, calcium iodide, calcium sulfate, and mixtures thereof are particularly preferred.

Following application of the water washing solution, impurities, unreacted components, and reaction byproducts are collected and removed from the washed reaction product. The washed reaction product separates into two discrete layers. The bottom layer contains the impurities, any solvent, reaction byproducts, and unreacted reaction components to be removed and discarded. The top layer contains the moderately esterified polyol fatty acid polyester. Optionally, the bottom layer may be collected and processed to recover and/or recycle any desired reaction ingredients and/or byproducts (e.g., polyol and solvent).

Separation into the discrete phases may be accomplished by allowing the washed reaction products to gravity settle. Preferred methods for the separation and isolation of impurities include centrifugation for a period of time in the range of from about 5 minutes to about 30 minutes at an applied force of from about 100 G to about 15000 G.

The various techniques for the isolation and removal of impurities and unwanted reaction byproducts described herein may be used either independently or in combination. In one embodiment of the present invention isolation of impurities occurs by centrifugation. In another embodiment, a product purification cycle comprising the steps of washing the reaction product with a solvent free water washing solution and then centrifuging the washed reaction product to isolate impurities is repeated for a total of ten times.

The purification process of washing the reaction product and separating and collecting the moderately esterified polyol polyester may optionally be performed one or more additional times, depending on product composition at the end of the purification cycle and the desired finished product purity specification. Preferably the purification cycle is repeated in the range of from about 1 to about 20 times to achieve particularly high degrees of purification.

In one embodiment of the present invention the water washing purification steps are repeated in the range of from about 2 to about 10 times. The quantity of water washing solution to be used in each purification cycle is calculated based on the initial weight of the reaction product to be purified (i.e., the weight of the reaction product prior to the first purification cycle). In each cycle the weight ratio of the water washing solution to the initial weight of the washed reaction product to be purified (e.g. initial reaction product; secondary reaction product; acid neutralized initial reaction product; or acid neutralized secondary reaction product) is within the range of from about 0.01:1 to about 1:1, preferably in the range of from about 0.05:1 to about 0.5:1, more preferably in the range of from about 0.1:1 to about 0.3:1.

The quantity of water washing solution utilized may be substantially the same for each purification cycle, or alternatively may vary from cycle to cycle. Additionally, the quantity of salt, if utilized in the water wash solution, may be substantially the same for each purification cycle, or alternatively may vary from cycle to cycle. Combinations of varying amounts of water and/or salt, if utilized, within the water washing solution of various purification cycles are also contemplated.

In one embodiment, the quantity of salt utilized in the water washing solutions of a purification cycle subsequent to the first purification cycle is less than the quantity of salt utilized in the previous purification cycle. In another embodiment, the quantity of salt utilized in the water washing solutions of a purification cycle subsequent to the first purification cycle is greater than the quantity of salt utilized in the previous purification cycle.

For each of the purification cycles the temperature of the water washing solution is in the range of from about 20° C. to about 100° C., and the temperature of the reaction product to be purified is in the range of from about 20° C. to about 100° C.

Optionally, the weight ratio of water washing solution to reaction product to be purified may be recalculated after each purification cycle, such that the weight ratio of the water washing solution to the weight of the reaction product to be purified in a given purification cycle is in the range of from about 0.01:1 to about 1:1, preferably in the range of from about 0.05:1 to about 0.5:1, more preferably in the range of from about 0.1:1 to about 0.3:1.

(b) Alcohol Purification Processes

The reaction products of the present invention may optionally be purified by an alcohol purification process, via application of an alcohol washing solution. Applicants have found that in order to obtain moderately esterified polyol polyester compositions with the requisite degree of purity, the alcohol purification process should be free of any additional solvents that would adversely affect the finished product purity requirement for the composition's intended use. As any solvent added after formation of the secondary reaction product must ultimately be removed via a purification process, it is preferred that the alcohol washing solution contain no additional ingredients that would not be substantially removed, preferably completely removed, by the alcohol wash process. Particularly preferred embodiments of the resent invention are those where the alcohol wash solution comprises no ingredients, other than perhaps impurities at a level that would not adversely impact finished product purity, beyond the alcohol.

The alcohol washing solution comprises alcohols with a carbon chain length in the range of from about 2 atoms to about 5 atoms. The alcohol washing solution is applied over a period of time in the range of from about 2 minutes to about 30 minutes, preferably in the rang of from about 5-10 minutes. The weight ratio of the alcohol washing solution to the initial weight of the reaction product to be purified (e.g. initial reaction product; secondary reaction product; acid neutralized initial reaction product; or acid neutralized secondary reaction product) is in the range of from about 0.01:1 to about 1:1, preferably in the range of from about 0.05:1 to about 0.5:1, more preferably in the range of from about 0.1:1 to about 0.3:1.

The temperature of the alcohol washing solution is in the range of from about 20° C. to about 100° C., and the temperature of the reaction product to be purified is in the range of from about 20° C. to about 100° C. Preferably the temperature of the alcohol washing solution is in the range of from about 20° C. to about 60° C. when the majority of the fatty acid esters are unsaturated, and in the range of from about 40° C. to about 80° C. when the majority of the fatty acid esters are saturated.

Examples of alcohols suitable for use in the present invention include ethanol, n-propanol, n-butanol, n-pentanol, branched and non-terminal forms of C₂-C₅ alcohols, and mixtures thereof. Preferred alcohols are selected from ethanol, n-propanol, n-butanol, n-pentanol, and mixtures thereof.

Following application of the alcohol washing solution, impurities, unreacted components, and reaction byproducts are collected and removed from the washed reaction product. The washed reaction product separates into two discrete layers. The bottom layer contains the impurities, any reaction solvent, reaction byproducts, and unreacted reaction components to be removed and discarded. The top layer contains the moderately esterified polyol fatty acid polyester. Optionally, the bottom layer may be collected and processed to recover and/or recycle any desired reaction ingredients and/or byproducts (e.g., polyol and/or solvent).

Separation into the discrete phases may be accomplished by allowing the impurities and byproducts to gravity settle. Preferred methods for the separation and isolation of impurities include centrifugation for a period of time in the range of from about 5 minutes to about 30 minutes at an applied force of from about 100 G to about 15000 G, preferably in the range of from about 2,000 G to about 10,000 G.

The purification cycle of washing the reaction product with alcohol and separating and collecting the moderately esterified polyol polyester may optionally be performed one or more additional times, depending on the product composition following the purification cycle and the desired degree of purity in the finished product. Preferably the purification process is repeated in the range of from about 1 to about 20 times to achieve particularly high degrees of purification.

In one embodiment of the present invention the alcohol washing purification steps are repeated in the range of from about 2 to about 10 times. The quantity of alcohol washing solution to be used in each purification cycle is calculated based on the initial weight of the reaction product to be purified (i.e., the weight of the reaction product prior to the first purification cycle). In each cycle the weight ratio of the alcohol washing solution to the initial weight of the washed reaction product to be purified (e.g. initial reaction product; secondary reaction product; acid neutralized initial reaction product; or acid neutralized secondary reaction product) is within the range of from about 0.01:1 to about 1:1, preferably in the range of from about 0.05:1 to about 0.5:1, more preferably in the range of from about 0.1:1 to about 0.3:1. The quantity of alcohol washing solution utilized may be substantially the same for each purification cycle, or alternatively may vary from cycle to cycle.

For each of the purification cycles the temperature of the alcohol washing solution is in the range of from about 20° C. to about 100° C., and the temperature of the reaction product to be purified is in the range of from about 20° C. to about 100° C.

Optionally, the weight ratio of alcohol washing solution to reaction product to be purified may be recalculated after each purification cycle, such that the weight ratio of the alcohol washing solution to the weight of the reaction product to be purified in a given purification cycle is in the range of from about 0.01:1 to about 1:1, preferably in the range of from about 0.05:1 to about 0.5:1, more preferably in the range of from about 0.1:1 to about 0.3:1.

(c) Drying

Optionally, the purified moderately esterified polyol polyester fatty acid compositions of the present invention may be dried by a variety of water or alcohol removal techniques commonly known to those ordinarily skilled in the art. A preferred drying technique employed in the processes of the present invention involves evaporation.

The purified, dried reaction product is formed by reacting the purified reaction product at a pressure in the range of from about 0.01 mmHg to about 760 mmHg, preferably in the range of from about 0.1 mmHg to about 20 mmHg, more preferably in the range of from about 0.1 mmHg to about 10 mmHg, most preferably in the range of from about 0.1 mmHg to abut 5 mmHg, and for a period of time in the range of from about 1 minutes to about 4 hours. One of ordinary skill in the art will appreciate upon reading the disclosure herein that the temperatures disclosed in the preferred temperature-pressure combinations refer to the temperature of the reaction ingredients, not the temperature setting of the equipment used to heat the reaction components. Subsequent to drying, the purified moderately esterified polyol polyester fatty acid compositions of the present invention that have been purified using water washing will have a Carl Fischer moisture content (as measured on a model MKA-5 ION Carl Fischer Moisture Titrator, produced by the Kyoto Electric manufacturing Company of Kyoto, Japan) of less than about 5%, preferably less than about 3%, more preferably less than about 1%, yet more preferably less than about 0.5%.

C. Composition of Purified, Moderately-Esterified Polyol Fatty Acid Polyesters

The purified, moderately esterified polyol polyester fatty acid compositions of the present invention generally comprise a moderately esterified polyol polyester with a degree of esterification of from about 40% to about 80%. Additionally, the purified, moderately esterified polyol polyester fatty acid compositions comprise less than about 5% polyol, preferably less than about 3.5% polyol, more preferably less than about 2% polyol, more preferably less than about 1.1% polyol; and is substantially free of residual solvent. As used herein, “substantially free of residual solvent” refers to a moderately esterified polyol polyester fatty acid composition comprising less than 5 ppm (parts per million) of residual solvent, alternatively less than about 4 ppm of residual solvent, alternatively less than about 3 ppm residual solvent, alternatively less than about 2 ppm residual solvent, preferably 0 ppm of residual solvent. Additionally, the purified, moderately esterified polyol polyester fatty acid compositions comprise less than about 700 ppm of lower alkyl esters, alternatively less than about 650 ppm of lower alkyl esters, alternatively less than about 500 ppm of lower alkyl esters, alternatively less than about 200 ppm of lower alkyl esters, alternatively less than about 100 ppm of lower alkyl esters, alternatively less than about 50 ppm of lower alkyl esters. Moreover, the purified, moderately esterified polyol polyester compositions comprise less than about 5% of a soap and free fatty acid mixture, alternatively less than about 4.5% of a soap and free fatty acid mixture, alternatively less than about 4% of a soap and free fatty acid mixture, alternatively less than about 3.5% of a soap and free fatty acid mixture, preferably less than about 1% of a soap and free fatty acid mixture.

The purified, moderately esterified polyol polyesters also comprise less than about 3% ash, preferably less than about 2% ash, more preferably less than about 0.5% ash. As used herein, the term “ash” refers to sulfated ash. The amount of sulfated ash in the present invention is calculated by weighing 5 grams of a sample into a platinum dish. Then 5 mL of 10% Sulfuric acid (H₂SO₄) is added to the sample, and the mixture is heated until carbonized. The carbonized ash is then baked in a muffle furnace at 550 C. until ashed. An additional aliquot of 2-3 mL of 10% Sulfuric Acid is added, and the mixture is again heated until carbonized. Again the mixture is baked at 550 C. until ashed. This process is repeated until the ash maintains a constant weight. The percentage of sulfated ash is calculated by dividing the weight of the remaining ash by the sample weight.

Furthermore, the purified polyester compositions of the present invention have an acid value of less than about 4, preferably an acid value less than about 3, more preferably an acid value less than about 2, most preferably an acid value less than about 0.5.

Not to be limited by theory, applicants believe residual levels of lower alkyl ester impurities may be attributed to those amounts that exist as an impurity within the highly esterified polyol polyester fatty acids prior to inclusion in the initial reaction mixture. Soap and free fatty acid mixtures are believed to be byproducts resulting from polyol degradation and catalyst neutralization reactions. Ash is also believed to be a byproduct of various degradation and purification processes within the synthesis of the purified, moderately esterified polyol polyester compositions.

Typically and preferably the purified polyester compositions of the present invention are light to clear in color. As measured on a Lovibond Model PFX995 Colorimeter, (Manufactured by Tintometer Ltd., The Colour Laboratory of Salisbury, UK) the purified compositions of the present invention have a Lovibond Red Color measurement of less than about 20, preferably less than about 15, more preferably less that about 10, yet more preferably less than about 5.

### D. EXAMPLES

The following are non-limiting examples of moderately esterified polyol polyester and purified, moderately esterified polyol polyester compositions and methods of making the same, used in accordance with the present invention. The following examples are provided to illustrate the invention and are not intended to limit the spirit or scope thereof in any manner.

### Example 1

In the present example, an initial reaction mixture comprises 1000 g (0.418 moles) of sucrose polyester, based on oleic fatty acids, with a degree of esterification of 96%; 9.3 g (0.027 moles) of powdered sucrose; 100 g (0.0616 moles) of moderately esterified sucrose polyester, based on oleic fatty acids, with an average degree of esterification of 62.5%, and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by super fluid chromatography (SFC) and found to have the composition shown in Table 1A, wherein SEₓ indicates a Sucrose Ester with x esterified hydroxyl groups. Suitable super fluid chromatography analytical methods are described in U.S. Pat. No. 6,566,124, issued May 20, 2003 to Trout et al., entitled Improved Processes for Synthesis and Purification of Nondigestible Fats. The table below represents the weight percents of the various sucrose esters on a solvent-free basis.

TABLE 1A

| Soap | Sucrose | SE₁ | SE₂ | SE₃ | SE₄ | SE₅ | SE₆ | SE₇ | SE₈ |
|---|---|---|---|---|---|---|---|---|---|
| 0.4 | — | — | — | — | 0.5 | 3.5 | 20.0 | 39.9 | 35.7 |

This represents a degree of esterification of about 87%.

### Example 2

In the present example, an initial reaction mixture comprises 1000 g (0.418 moles) of sucrose polyester, based on oleic fatty acids, with a degree of esterification of 96%; 41.7 g (0.122 moles) of powdered sucrose; 200 g (0.106 moles) of moderately esterified sucrose polyester, based on oleic fatty acids, with an average degree of esterification of 75%, and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 2A.

TABLE 2A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | — | — | — | 0.7 | 5.5 | 21.0 | 36.0 | 28.0 | 8.4 |

This represents a degree of esterification of about 75%.

The initial reaction product is then neutralized using 7.0 g of 36.5% hydrochloric acid in water.

The neutralized initial reaction product is then purified with 109 g of deionized water. This water wash is carried out at 60° C. under mild agitation for 10 minutes. This purified product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.2% A sample of the purified, dried reaction product from the evaporation is retained and any water and/or volatile impurities from the evaporator can be collected and recycled.

A sample of the purified, dried reaction product is analyzed by Super Fluid Chromatography (SFC) and found to have the composition shown in Table 2B.

TABLE 2B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | — | — | — | 0.8 | 5.4 | 21.2 | 35.9 | 28.1 | 8.2 |

### Example 3

In the present example, an initial reaction mixture comprises 1000 g (0.418 moles) of sucrose polyester, based on oleic fatty acids, with a degree of esterification of 96%; 78.5 g (0.229 moles) of powdered sucrose; 400 g (0.247 moles) of moderately esterified sucrose polyester, based on oleic fatty acids, with an average degree of esterification of 62.5%, and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 3A.

TABLE 3A

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | — | 0.1 | 1.4 | 7.0 | 19.5 | 32.6 | 27.7 | 9.6 | 1.3 |

This represents a degree of esterification of about 62%.

The initial reaction product is then purified with 170 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.1%. A sample of the purified, dried reaction product from the evaporator is retained and any water and/or other volatile impurities from the evaporator can be collected and recycled.

A sample of the purified, dried reaction product from the evaporator is analyzed by Super Fluid Chromatography (SFC) and found to have the composition shown in Table 3B.

TABLE 3B

| Soap | Sucrose | SE₁ | SE₂ | SE₃ | SE₄ | SE₅ | SE₆ | SE₇ | SE₈ |
|---|---|---|---|---|---|---|---|---|---|
| 0.5 | — | 0.1 | 1.4 | 7.2 | 19.3 | 32.7 | 27.9 | 9.3 | 1.3 |

### Example 4

In the present example, an initial reaction mixture comprises 1000 g (0.418 moles) of sucrose polyester, based on oleic fatty acids, with a degree of esterification of 96%; 160 g (0.469 moles) of powdered sucrose; 500 g (0.308 moles) of moderately esterified sucrose polyester, based on oleic fatty acids, with an average degree of esterification of 62.5%, and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 4A.

TABLE 4A

| Soap | Sucrose | SE₁ | SE₂ | SE₃ | SE₄ | SE₅ | SE₆ | SE₇ | SE₈ |
|---|---|---|---|---|---|---|---|---|---|
| 0.6 | — | 0.7 | 5.9 | 17.7 | 32.2 | 29.3 | 12.1 | 1.5 | 0.0 |

This represents a degree of esterification of about 50%.

The initial reaction product is then purified with 150 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.2%. A sample of the purified, dried reaction product from the evaporation is retained and any water and/or other volatile impurities from the evaporator can be collected and recycled.

A sample of the purified, dried reaction product from the evaporator is analyzed by Super Fluid Chromatography (SFC) and found to have the composition shown in Table 4B.

TABLE 4B

| Soap | Sucrose | SE₁ | SE₂ | SE₃ | SE₄ | SE₅ | SE₆ | SE₇ | SE₈ |
|---|---|---|---|---|---|---|---|---|---|
| 0.6 | — | 0.8 | 5.9 | 17.8 | 32.0 | 29.2 | 12.3 | 1.4 | 0.0 |

### Example 5

In the present example, an initial reaction mixture comprises 1000 g (0.418 moles) of sucrose polyester, based on oleic fatty acids, with a degree of esterification of 96%; 268 g (0.784 moles) of powdered sucrose; 500 g (0.366 moles) of moderately esterified sucrose polyester, based on oleic fatty acids, with an average degree of esterification of 50%, and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 5A.

TABLE 5A

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.1 | 19.8 | 33.5 | 28.0 | 10.4 | 1.8 | 0.0 | 0.0 |

This represents a degree of esterification of about 37.5%.

The initial reaction product is then purified with 150 g of deionized water. This water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.3%. A sample of the purified, dried reaction product from the evaporation is retained and any water and/or other volatile impurities from the evaporator can be collected and recycled.

A sample of the purified, dried reaction product from the evaporator is analyzed by Super Fluid Chromatography (SFC) and found to have the composition shown in Table 5B.

TABLE 5B

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.2 | 20.0 | 33.3 | 28.0 | 10.4 | 1.8 | 0.0 | 0.0 |

The purified, dried reaction product has an acid value of about 0.4, a lower alkyl ester level of about 250 ppm, an ash level of about 0.1%, and no residual reaction solvent.

### Example 6

An initial reaction mixture comprises 1000 g (0.418 moles) of sucrose polyester, based on oleic fatty acids, with a degree of esterification of 96%; 268 g (0.784 moles) of powdered sucrose; 500 g (0.366 moles) of moderately esterified sucrose polyester, based on oleic fatty acids, with an average degree of esterification of 50%, and 10 g (0.072 moles) of potassium carbonate. Prior to use in the initial reaction mixture the sucrose and catalyst are dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 6A.

TABLE 6A

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.1 | 19.8 | 33.5 | 28.0 | 10.4 | 1.8 | 0.0 | 0.0 |

This represents a degree of esterification of about 37.5%.

The initial reaction product is then purified with 150 g of methanol. This alcohol wash is carried out at 50° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a methanol content of about 0.1%. A sample of the purified, dried reaction product from the evaporation is retained and any methanol and/or other volatile impurities from the evaporator can be collected and recycled.

A sample of the purified, dried reaction product from the evaporator is analyzed by Super Fluid Chromatography (SFC) and found to have the composition shown in Table 6B.

TABLE 6B

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.2 | 20.0 | 33.3 | 28.0 | 10.4 | 1.8 | 0.0 | 0.0 |

The purified, dried reaction product has an acid value of about 0.5, a lower alkyl ester level of about 300 ppm, an ash level of about 0.2%, and no residual reaction solvent.

### Example 7

In the present example, an initial reaction mixture comprises 1000 g (0.418 moles) of sucrose polyester, based on oleic fatty acids, with a degree of esterification of 96%; 268 g (0.784 moles) of powdered sucrose; 500 g (0.366 moles) of moderately esterified sucrose polyester, based on oleic fatty acids, with an average degree of esterification of 50%, and 10 g (0.072 moles) of potassium carbonate. Prior to use in the initial reaction mixture the sucrose and catalyst are dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by Super Fluid Chromatography (SFC) and found to have the composition shown in Table 7A.

TABLE 7A

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.1 | 19.8 | 33.5 | 28.0 | 10.4 | 1.8 | 0.0 | 0.0 |

This represents a degree of esterification of about 37.5%.

The initial reaction product is then purified with 150 g of a 1% NaCl solution in deionized water. This salt/water wash is carried out at 60° C. under mild agitation for 10 minutes. This purified reaction product is then centrifuged and the top product layer is decanted and the bottom water layer is discarded. The top product layer is then dried on a wiped film evaporator operating at 100° C. and 1 mmHg with a residence time of about 2 minutes. The purified, dried reaction product has a moisture content of about 0.1%. A sample of the purified, dried reaction product from the evaporator is retained and any water and/or volatile impurities from the evaporator can be collected and recycled.

A sample of the purified, dried reaction product from the evaporator is analyzed by Super Fluid Chromatography (SFC) and found to have the composition shown in Table 7B.

TABLE 7B

| Soap | Sucrose | SE₁ | SE₂ | SE₃ | SE₄ | SE₅ | SE₆ | SE₇ | SE₈ |
|---|---|---|---|---|---|---|---|---|---|
| 0.7 | 0.7 | 5.2 | 20.0 | 33.3 | 28.0 | 10.4 | 1.8 | 0.0 | 0.0 |

### Example 8

In the present example, an initial reaction mixture comprises 1000 g (0.418 moles) of sucrose polyester, based on oleic fatty acids, with a degree of esterification of 96%; 9.3 g (0.027 moles) of powdered sucrose; 100 g (0.0616 moles) of moderately esterified sucrose polyester, based on oleic fatty acids, with an average degree of esterification of 62.5%; 50 g of dimethyl sulfoxide solvent; and 10 g (0.072 moles) of potassium carbonate. Prior to use in the initial reaction mixture the sucrose and catalyst were dried in a vacuum oven for 12 hours. An initial reaction product is formed by reacting the initial reaction mixture at 100° C. for 300 minutes in a two-piece, baffled glass reactor. The initial reaction mixture is reacted in the presence of agitation to ensure even heat distribution of the reaction components.

A sample of the initial reaction product is analyzed by super fluid chromatography (SFC) and found to have the composition shown in Table 8A, wherein SEX indicates a Sucrose Ester with x esterified hydroxyl groups. Suitable super fluid chromatography analytical methods are described in U.S. Patent U.S. Pat. No. 6,566,124, issued May 20, 2003 to Trout et al., entitled Improved Processes for Synthesis and Purification of Nondigestible Fats. The table below represents the weight percents of the various sucrose esters on a solvent-free basis.

TABLE 8A

| Soap | Sucrose | SE₁ | SE₂ | SE₃ | SE₄ | SE₅ | SE₆ | SE₇ | SE₈ |
|---|---|---|---|---|---|---|---|---|---|
| 0.4 | — | — | — | — | 0.5 | 3.5 | 20.0 | 39.9 | 35.7 |

This represents a degree of esterification of about 87%.

1. A purified, moderately esterified polyol fatty acid polyester composition comprising:
i) a moderately esterified polyol fatty acid polyester;
ii) less than about 5% polyol;
iii) less than about 700 ppm of lower alkyl esters;
iv) less than about 2% of a soap and free fatty acid mixture;
v) less than about 1% of ash; and
wherein the polyester composition is substantially free of residual solvent; and
wherein the polyester composition has an acid value of less than about 2.

2. The composition of claim 1 wherein said polyol polyester composition has a degree of esterification of from about 40% to about 80%.

3. The composition of claim 2 wherein to about 70%.

4. The composition of claim 3 wherein said polyol polyester composition has a degree of esterification that is about 60%.

5. The composition of claim 1 wherein said residual solvent is selected from dimethyl sulfoxide, n-methyl formamide, dimethyl sulfate, formamide, and mixtures thereof.

6. The composition of claim 5 wherein said residual solvent is dimethyl sulfoxide.

7. The composition of claim 1 where the level of residual solvent present in the polyol polyester composition is 0 ppm.

8. The composition of claim 1 wherein the lower alkyl ester is selected from methyl esters, ethyl esters, propyl esters, butyl esters, and mixtures thereof.

9. The composition of claim 1 wherein said lower alkyl ester is methyl ester.

10. The composition of claim 1 wherein said purified, moderately esterified polyol fatty acid polyester is a sucrose fatty acid polyester.

11. The composition of claim 1 wherein said composition comprises less than about 2% of said polyol, no residual solvent, less than about 600 ppm of said lower alkyl esters, less than about 1% of said soap and fatty acid mixture, less than about 0.5% of said ash, and said acid value is less than about 1.

12. The composition of claim 11 wherein said purified, moderately esterified polyol fatty acid polyester is a sucrose fatty acid polyester and said polyol is sucrose.

13. A purified, moderately esterified sucrose fatty acid polyester composition comprising:
i) a moderately esterified sucrose fatty acid polyester;
ii) less than about 5% sucrose;
iii) less than about 3 ppm of residual solvent;
iv) less than about 700 ppm of lower alkyl esters;
v) less than about 2% of a soap and free fatty acid mixture;
vi) less than about 1% of ash; and,
wherein the moderately esterified sucrose fatty acid polyester composition has an acid value of less than about 2.

14. The composition of claim 13 wherein said composition comprises less than about 1% of said sucrose, 0 ppm of residual solvent, less than about 600 ppm of said lower alkyl esters, less than about 1% of said soap and fatty acid mixture, less than about 0.5% said ash, and has an acid value of less than about 1.

15. A lubricant composition comprising the purified, moderately esterified polyol polyester composition of claim 1.

16. A laundry composition comprising the purified, moderately esterified polyol polyester composition of claim 1.

17. A cosmetic composition comprising the purified, moderately esterified polyol polyester composition of claim 1.

18. A food composition comprising the purified, moderately esterified polyol polyester composition of claim 1.

19. The food composition of claim 18 wherein said purified, moderately esterified polyol fatty acid composition is a purified, moderately esterified sucrose fatty acid composition, said polyol is sucrose, said solvent is dimethyl sulfoxide, and said lower alkyl esters are selected from methyl esters, ethyl esters, and mixtures thereof.

20. A laundry composition comprising the purified moderately esterified polyol polyester composition of claim 13.

21. A cosmetics composition comprising the purified moderately esterified polyol polyester composition of claim 13.

22. The food composition of claim 18 wherein said purified, moderately esterified polyol fatty acid composition is a purified, moderately esterified sucrose fatty acid composition, said polyol is sucrose, no solvent is present, and said lower alkyl esters are selected from methyl esters, ethyl esters, and mixtures thereof.
